(19) United States
(12) Patent Application Publication
(10) Pub. No.: US 2007/0172943 A1
(43) Pub. Date: Jul. 26, 2007

(54) DEVICES AND METHODS FOR CULTURING, PRESERVING, TRANSPORTING, STORING, RECONSTITUTING AND ASSAYING CELLULAR MATERIALS

(76) Inventors: Robert Freedman, Beverly Hills, CA (US); Martin Yarmush, Newton, MA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/556,956

(22) Filed: Nov. 6, 2006

Related U.S. Application Data
(60) Provisional application No. 60/734,066, filed on Nov. 7, 2005.

(51) Int. Cl. C12M 3/00

(52) U.S. Cl. 435/293.1

(57) ABSTRACT

Devices, systems and methods for preparing, packaging and using biological materials are disclosed. Biological materials such as cellular or subscellular materials can be introduced in one or more complete assembled devices and preserved within the devices by, for example, being cooled or frozen. Such assembled devices can allow easy transportation and subsequent storing of the biological materials. In some embodiments, the assembled devices can be configured to provide for flow of one or more fluids. Thus, the biological materials can be preserved or reconstituted for use by being in contact with the fluids or by being warmed by the fluid(s), which may be flowing. In one embodiment, subsequent assays using the biological materials in the same assembled device(s) wherein their preparation or packaging previously occurred can involve fluidic interactions among the biological materials or between the biological materials and the fluid or its contents. In one embodiment, one or more features of the assembled device for biological material storage and/or fluid flow can be fabricated in microscale. Larger-scale features and/or devices are also possible.

### PRIORITY CLAIM

This application claims the benefit of priority under 35 U.S.C. § 119(e) of U.S. Provisional Application No. 60/734,066 filed on Nov. 7, 2005 and titled DEVICES AND METHODS FOR TRANSPORTING, STORING, RECONSTITUTING AND ASSAYING CELLULAR MATERIALS, the entirety of which is incorporated herein by reference.

### BACKGROUND

1. Field

The present disclosure relates to cellular material technology, and more particularly, to systems and methods for culturing, preserving, transporting, storing, reconstituting, and assaying cellular material.

2. Description of the Related Art

In vitro cell culture assays can provide valuable tools for researchers for studies in areas such as drug discovery and development, environmental testing, nutritional development and testing, consumer and industrial product safety, toxicology, and clinical diagnostics and treatments. Data from such in vitro studies can be extrapolated for applications to animals and humans. Biological materials associated with such studies pose various challenges associated with their culture, preservation, storage, transportation, and other processing or assaying steps. There is a need and an opportunity to provide devices and methods that offer advancements and innovations over the existing state of the related art, in terms of providing increased convenience of materials handling, and also in terms of processing biological materials so as to enable them to maintain or recover more of the functionality that they may naturally manifest in vivo, which functionality is often compromised under the current state of the related art; or to develop such other functionalities as may be of interest.

### SUMMARY

In one embodiment, a microscale in vitro culture device comprised in combination with added cellular and/or extracellular material is described. Microscale means that the device is configured to have at least one feature with at least one dimension measuring between 0.1 micrometer (“micron,” “μm”) and 1 centimeter (“cm”). In one embodiment, larger scaled features and/or devices providing similar functionalities are possible.

In one embodiment, the culture device is configured to comprise one or more discrete culture chambers; one or more channels to transport or hold fluid entering into or connecting to or between the one or more culture chambers, thus defining a culture system; and at least one type of cellular material such as cells, tissues, subcellular components, or cellular products (whether any of the foregoing may be primary, artificial, synthetic, engineered, or any combination thereof), which cellular material is an integral part of the disclosure and which may be cultured in, on, or with at least one type of matrix material such as an extracellular matrix material such as collagen, fibrinogen or MATRIGEL or a hydrogel matrix material such as calcium alginate or gelatin, or the like, which matrix material, if any, can also be an integral part of the disclosure. The culture device may be mounted or integrally formed inside a housing which, when sealed, enables the culture system to be a closed system. The housing may also comprise additional features or functions integrated within it such as fluid interconnects, electronic circuitry, sensors, transducers, microprocessors, temperature or atmospheric conditioners, interfaces to other devices and systems including without limitation the internet, pumps or other fluid actuators, debubblers, reservoirs, assays, and the like. The cellular materials and/or matrix materials that may be comprised in a particular embodiment of the disclosure can be collectively referred to herein as the “biological materials”; and the culture device comprised all-inclusively with its housing, if any, and with its added biological materials can be referred to herein as a “Complete Assembled Device” (“CAD”).

In one embodiment, the housing may also be configured to comprise at least one input and at least one output that enables the flow of fluid between and among the Complete Assembled Device and one or more of external tubing, pumps, sensors, transducers, heaters, coolers, atmospheric control devices, gas exchange devices, fluid reservoirs, debubblers, interfaces to other bioanalytic devices, interfaces to automated materials handling equipment, interfaces to computers, microprocessors, or the internet, or other materials or devices which, in conjunction with the Complete Assembled Device, comprise a system. In an alternative embodiment, any or all the foregoing elements of the system may be comprised integrally with the housing.

Biological materials may comprise types or sources of cellular material such as tissues, tissue slices or samples, primary cells, cell lines, transfected cells, reporter cells, and the like; they may also comprise subcellular material. The cellular material or the subcellular material can be either naturally occurring or man-made. Subcellular material can be subcellular components, such as mitochondria, microsomes and the like. For example, a chamber designed to enable liver enzyme metabolic activity might be configured to culture, preserve, store, maintain, reconstitute, or recover at least one of the physioogical attributes or functions of isolated liver microsomes. Alternatively, subcellular material can be cellular products, such as enzymes, nucleic acids, and the like. For example, a chamber designed to enable liver cytochrome P450 enzyme activity might be configured to culture, preserve, store, maintain, reconstitute, or recover at least one of the physiological attributes or functions of immobilized liver cytochrome P450 enzyme(s).

The cellular products can be derived from an appropriate mammanlian cell or they can be synthetic. An example of a synthetic cellular product would be an enzyme which differs in structure and/or activity from the naturally occurring enzyme through a process of genetic manipulation or chemical synthesis. The subcellular components can be dervied from an appropriate mammalian cell or they can be synthetic. An example of a synthetic subcellular component would be an artificial microsome.

The biological materials may be cultured, assembled, fixed, stabilized, adhered or preserved in one or more of the chambers or channels of the culture device, causing the Complete Assembled Device to advantageously enable the storage, physical transport, and/or further processing of the biological materials prior to their subsequent extraction from the Complete Assembled Device or prior to their employment in a biological assay, as part of a bio-reactor or culture system, or in some other form of experiment or procedure, whether for bioanalytic, toxicological, diagnostic, thereapeutic, or other purposes, while maintained in the Complete Assembled Device. The biological materials may be added to and maintained in the culture device in substantially the same physiological state as that in which they existed in vivo or in vitro prior to their addition to the culture device. Alternatively, the biological materials may be processed prior to, during, or after their addition to the culture device such that at least one aspect of their physiological state changes from what it previously was either in vivo or in vitro prior to their addition to the culture device; and/or such that a further process is required to reconstitute, recover, restore, or further change at least one aspect of their physiological function or state prior to the biological materials' being utilized in an assay, as part of a bio-reactor, or in some other experiment or process.

Upon the introduction of at least one fluid (liquid or gas) which enters into, diffuses either directly or through a permeable membrane into another material already present in, partially or completely fills, circulates or recirculates through, intermittently or continuously remains in, is subsequently evacuated from, or is subsequently diminished, diluted, displaced or replaced by the introduction of one or more different materials into, the one or more microfluidic chambers and channels of the culture system, which fluid thereby comes into contact with at least some of the biological materials and thereby comprises a culture medium, the Complete Assembled Device advantageously enables cellular material cultured in vitro to preserve, maintain, recover, or perform at least one physiological attribute or function in a manner, to a degree, or over a duration of time that is either more closely simulative of how that same cellular material preserves, maintains or performs that physiological attribute or function in vivo (compared to how that cellular material maintains or performs that same attribute or function in vitro in the Complete Assembled Device in the absence of any introduction of that same fluid), or that more perfectly attains some other attribute or function that the operator(s) of the Complete Assembled Device may desire or specify the biological materials to attain (compared to how that cellular material maintains or performs that same attribute or function in vitro in the Complete Assembled Device in the absence of any introduction of that same fluid). The flow or other movement of the culture medium into, within, or out of the Complete Assembled Device may advantageously enable the preservation of the cellular materials, or the recovery or reconstitution of the at least one physiological attribute or function in vivo (compared to how that cellular material maintains or performs that same attribute or function in vitro in the Complete Assembled Device in the absence of any flow or other movement of that same culture medium); or may cause the more perfect attainment of some other attribute or function that the operator(s) of the Complete Assembled Device may desire or specify the biological materials to attain (compared to how that cellular material maintains or performs that same attribute or function in vitro in the Complete Assembled Device in the absence of any flow or other movement of that same culture medium). The culture medium may comprise one or more solutes such as dimethyl sulfoxide (“DMSO”), glycerol, trehalose and the like, to serve as cryoprotectants, as molecular inhibitors of various damage pathways involving reactive oxygen species, apoptosis, and the like, or as membrane stabilizers. The Complete Assembled Device thereby advantageously enables biological materials including cellular materials to be utilized in biological assays, as part of a bio-reactor, or in other experiments or procedures after first having been cultured, assembled, fixed, stabilized, adhered, preserved, transported, stored, recovered or reconstituted in, and/or removed from, the culture device.

In one embodiment, the method of culturing, preserving, maintaining, storing, transporting, reconstituting and/or recovering at least one physiological attribute or function of the biological materials may comprise methods for cooling or freezing such as using controlled rate freezers, passive cooling, nucleation of extracellular ice, and using the liquid culture medium in the Complete Assembled Device as a circulating refrigerant. Alternatively, the foregoing methods of culturing, preserving, maintaining, storing, transporting, reconstituting and/or recovering may comprise mechanically inserting within the Complete Assembled Device biological materials that had previously been preserved, cryopreserved or cold-preserved externally to the Complete Assembled Device.

In one embodiment, the method of culturing, preserving, maintaining, storing, transporting, reconstituting and/or recovering at least one physiological attribute or function of the biological materials may comprise immobilizing cellular or subcellular material in extracellular matrix material such as a caclium alginate scaffold. The cellular or subcellular material may be preserved, cold-preserved or cryopreserved inside the calcium aliginate scaffold. The calcium alginate scaffold containing the immobilized, preserved cells is inserted into a chamber of the Complete Assembled Device. Alternatively, the cellular or subcellular material may be added to, immobilized, and preserved within the scaffold after the scaffold has already been placed or otherwise configured inside the chamber of the Complete Assembled Device. The Complete Assembled Device is thus configured to serve as the physical container or vehicle for the maintenance, storage, transportation, reconstitution, and/or recovery of at least one physiological attribute or function of the biological materials. The same Complete Assembled Device may also be configured to serve, and may then serve, as the physical container or vehicle in which at least one biological assay, experiment, bioreactor process, or diagnostic, therapeutic or other process is subsequently performed using the same or different biological materials as those that were previously therein cultured, preserved, maintained, stored, transported, or reconstituted, or those of which at least one physiological attribute or function was previously therein recovered. In an alternative embodiment, the cellular or subcellular material is immobilized and cryopreserved or cold-preserved inside the calcium alginate scaffold. The scaffold itself then becomes the mechanical container or vehicle for the maintenance and storage of the biological materials, and for the transportation of the biological materials to the location where they may be inserted into a Complete Assembled Device, their at least one physiological attribute or function may be recovered or reconstituted, and where the Complete Assembled Device may then be advantageously used in biological assays, as part of a bio-reactor, or in other experiments or procedures. In another embodiment, the extracellular matrix material is a hydrogel or a three-dimensional construct comprised of layers of collagen, Matrigel, biomatrix, or the like configured on top of and/or beneath a layer of cellular or subcellular material. The method of preserving, maintaining, storing, transporting, reconstituting and/or recovering at least one physiological attribute or function of the biological material may also comprise maintaining the cellular or subcellular materials in a particular configuration or shape, such as in a sphere. The method may also provide for the biological materials to be cultured, preserved, maintained, stored, transported, recovered and/or reconstituted under vacuum conditions, such as inside a bag or other packaging, container or housing that enables the maintainance of vacuum conditions within it. In another embodiment, the biological material is introduced, cultured, preserved, maintained, stored, or transported in the Complete Assembled Device by any means whatsoever; and the biological material is subsequently reconstituted, at least one physiological attribute or function of the biological material is subsequently recovered, or a biological assay, experiment, bioreactor procedure, or diagnostic, therapeutic or other procedure is subsequently performed, in the Complete Assembled Device wherein fluid culture medium is introduced into or is intermittently or continuously maintained in the Complete Assembled Device under a flow condition. In still another embodiment, biological material is cultured, preserved, maintained, stored, transported, reconstituted, or with respect to at least one physiological attribute or function recovered, in the Complete Assembled Device wherein the method of introducing fluid culture medium into, or intermittently or continuously maintaining fluid culture medium in, the Complete Assembled Device under a flow condition is employed in conjunction with at least one other method of culturing, preserving, maintaining, storing, transporting, reconstituting, or with respect to at least one physiological attribute or function recovering, the biological material.

### BRIEF DESCRIPTION OF THE DRAWINGS

FIG. 1 shows one embodiment of an assembled culture device being utilized in a fluidic system;

FIG. 2 shows a partially unassembled view of one embodiment of the assembled culture device; and

FIG. 3 shows one embodiment of a product kit that can include one or more of the assembled culture devices.

These and other aspects, advantages, and novel features of the present teachings will become apparent upon reading the following detailed description and upon reference to the accompanying drawings. In the drawings, similar elements have similar reference numerals.

### DETAILED DESCRIPTION OF SOME EMBODIMENTS

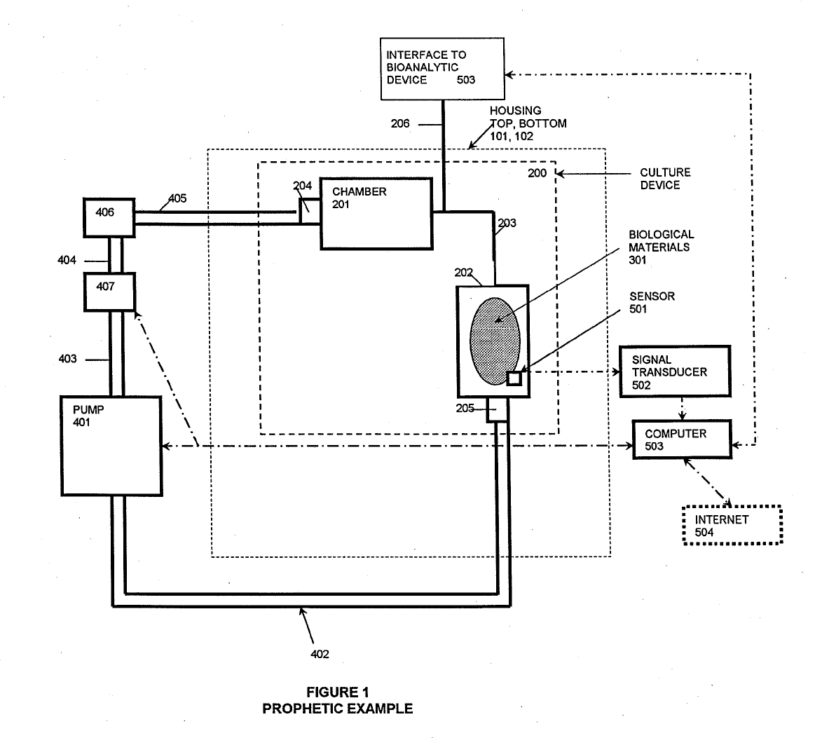

FIG. 1 schematically represents one embodiment of a prophetic example of the present disclosure. The culture device 200, comprised of one or more chamber(s) 201, 202, at least one channel 203 connecting to the at least one chambers, an inlet 204 and an outlet 205 for the flow of culture medium, is situated inside the housing top and housing bottom 101 and 102 respectively; i.e., the culture device comprises the inner portion of the device, while the housing top and bottom respectively comprise the top and bottom portions of the device. Biological materials 301, which may include cellular or subcellular materials as well as extracellular matrix materials such as a hydrogel, a gel material such as collagen, Matrigel, and the like placed above and or below the cellular or subcellular material in a layered configuration or alginate scaffolds that maintain the cellular or subcellular material in a three-dimensional configuration, are shown here cultured inside the one or more chambers 202. In some embodiments, the foregoing elements can be part of the Complete Assembled Device.

The prophetic example shown in FIG. 1 also shows how the CAD comprises an element of a system also comprising other materials and devices. This system also comprises a pump 401, which may be peristaltic, electrostatic, hydraulic, or any other kind of pump, to actuate the flow of culture medium through and from the culture device 200, through microfluidic tubing 402, 403, 404, and 405, through other mechanisms 406, 407 influencing the status or condition of the culture medium as described below, and back into the inlet 204 of the culture device that comprises the CAD; although in alternative embodiments the flow path of the culture medium would not recirculate as shown here, but rather would travel in a linear, non-recirculating pathway, still actuated by a pump, starting from one reservoir 406, traveling through the one or more chambers 201, 202 and one or more channels 203 of the culture device 200, and terminating in a second, separate reservoir 406. In the present prophetic example of FIG. 1, the microfluidic flow path outside of the culture device itself is configured to enable fluid culture medium to travel from the outlet 205, through the tubing 402, through the pump 401, through the tubing 403, through a fluidic temperature and/or atmospheric control device 407 which may at various times either heat or cool the culture medium, or provide for gas exchange or for regulation or modification of the constituents of the culture medium, through the tubing 404, through a reservoir 406 which is configured to provided for a larger volume of culture medium to operate in the device than will fit at any one time into the aggregate volume of all the other chambers, channels, and other fluid-holding features and elements of the system, and which reservoir 406 also comprises a sampling port, a mechanism for replenishing or adding to the contents of the culture medium, and a debubbler to remove bubbles from the flow path of the culture medium, and finally returning via the tubing 405 to the inlet 204 of the culture device. From there, the recirculatory flow path is completed as the culture medium travels from the inlet 204 into the at least one chamber 201, through the at least one channel 203, through the at least one chamber 202 wherein the culture medium comes in contact with the biological materials 301, and finally back again to the outlet 205.

The embodiment of a prophetic example shown in FIG. 1 also demonstrates that the CAD connects through a fluidic interface 203 to at least one separate bioanalytic device such as a mass spectroscopy device, a high-pressure liquid chromatography device, a fluourometric device, or the like. In this embodiment the at least one chamber 202 also comprises a sensor 501 which may be in contact with the biological material, the culture medium, or both. The information derived from the CAD by the at least one sensor is converted by an appropriate transducer 502 to an electrical signal readable and analyzable by a computer 503. Based on the data the computer 503 thus receives from the CAD, or based on other data the computer 503 may possess or receive, the computer may send and/or receive that data, or other information, to other devices or locations via telecommunications pathways such as the internet 504. The computer 503 may also provide information to other elements of the system, such as to the pump 401 and/or to the fluidic temperature or atmospheric control device 407 as shown here, or through the interface to one or more bioanalytic devices 503 to such one or more devices (not illustrated) or to other elements of the system, in order to control elemeents of the system or influence the state of the fluid culture medium or the biological materials 301.

In one embodiment, the chamber can contain a combination of cultured cells, subcellular components, and cellular products. The cultured cells may be all of the same type or they may comprise a co-culture of more than one type; the subcellular components may be all of the same type or they may comprise more than one type; the cellular products may be all of the same type or they may comprise more than one type.

In another embodiment, the chamber is also comprised of at least one inlet and at least one outlet for the flow of fluid respectively into and out from the chamber.

In another embodiment, the chamber is connected to multiple channels which may be configured to provide for the separate flow of different fluids within the device. For example, a chamber might be configured to connect to at least one channel which at certain times fluidically delivers to or evacuates from the chamber one or more fluids advantageously useful to the preservation, maintenance, storage, reconstitution or recovery of at least one physiological attribute or function of biological material in the Complete Assembled Device; and the chamber might be further configured to connect to a different at least one channel which at certain times delivers to or evacuates from the chamber liquid culture medium containing one or multiple drug candidate species, toxicants or potential toxicants, or other xenobiotic materials or species of interest that may come into contact with biological materials within the chamber or elsewhere within the Complete Assembled Device. In yet another embodiment, the Complete Assembled Device may be configured to convey different culture media at different times through the same at least one chamber or the same at least one channel. In another embodiment, a single culture medium may be comprised of materials that enable it to advantageously serve both as a facilitator of the preservation, storage, maintenance, reconstitution or recovery of at least one physiological attribute or function of the biological material, and also to serve as a medium that conveys drug candidate species, toxicants or potential toxicants, or other xenobiotic materials or species of interest and fluidically brings them into contact with the biological materials. In another embodiment, The Complete Assembled Device is configured to expose one or multiple drugs or other species of interest to biological materials maintained in one or multiple chambers of, or circulating within, the Complete Assembled Device.

In another embodiment, the chamber may contain a confluent monolayer of cells, such as gastrointestinal epithelial cells, such that fluid may flow along one or both sides of but not through the monolayer, and the intervening cell layer thus provides a barrier to fluid flow. In yet another embodiment, the barrier to fluid flow is a membrane, which may be comprised of naturally occurring materials, man-made or other artificially occurring materials, or a combination of the two types of materials. The membrane may be permeable to gas but not liquid or vice versa, it may permit the diffusion or other passive transport through the membrane of solid materials of one range of size, solubility, or permeability but not of a different such range, or it may be comprised of proteins such as polyglycoproteins and the like that selectively facilitate the active transport of certain materials through the membrane. In another embodiment, the fluid that flows along or is contiguous to one side of the membrane may be at least one liquid, while the fluid that flows along or is contiguous with the other side of the membrane may be at least one gas. In various, other embodiments of the present disclosure, the cells, cellular components, cellular products, or various combinations thereof as the case may be, may be adherent to the chamber or the membrane, or alternatively they may be free to circulate within the Complete Assembled Device; or alternatively, some may be adherent while others circulate.

Preservation and storage of biological materials in the Complete Assembled Device may be either for short term or for long term (for example, for less than forty-eight hours or for more than forty-eight hours), may be under conditions of hypothermia, cold preservation, cryopreservation, dehydration, dessication, or the like; and may occur at the place of assembly of the Complete Assembled Device, at the laboratory where the Complete Assembled Device is waiting to be placed into operation or where biological materials are extracted from it, at a separate storage location, at some other location, or at more than one of the foregoing. Physical transport of the Complete Assembled Device may occur between the original place of assembly, the laboratory where the Complete Assembled Device is placed into operation or where biological materials are extracted from it, a separate storage location, some other location, or between more than any two of the foregoing. Processing of the biological materials may occur before, during, and/or after their being added to the Complete Assembled Device. Maintenance or reconstitution of at least one physiological attribute of the biological material by means of introducing the fluid which flows into or through the Complete Assembled Device, thus serving as a culture medium, may occur during storage or transportation of the Complete Assembled Device, or during or prior to use of the Complete Assembled Device in the laboratory, or during more than one of any of the foregoing.

In one embodiment, the Complete Assembled device comprises a system wherein the at least one compartment or channel comprises a storage chamber that is advantageous for the physical transportation of the biological material from place to place. Comprising both the microfluidic circuit and at least some biological material, the same Complete Assembled Device may serve as both the shipping or transportation container or vehicle in which the biological material is transported, and then it is also used for the reconstitution of the biological material. The same Complete Assembled Device may be the vehicle for shipment and then for the reconstitution or recovery of at least one physiological attribute or function of the biological material, and for assay of the biological material. The same Complete Assembled Device may be the vehicle for shipment and then for assay of the biological material. In some embodiments, no more biological material is added to the Complete Assembled Device by the end user or some other party who receives the Complete Assembled Device after it has been transported. In other embodiments, additional biological material is added to the Complete Assembled Device by the end user or some other party who receives the Complete Assembled Device after it has been transported. The biological material may be adherent or non-adherent. It may or may not be perfused with cell culture medium which is recirculating continuously or intermittently during shipment. It may be cultured in a cellular matrix material such as one of the group of extracellular matrix materials such as collagen, fibrinogen or MATRIGEL; or one of the group of hydrogel matrix materials such as calcium alginate or gelatin, or the like; or in a cellular matrix material that is not a member of either of the aforementioned groups.

In one embodiment of the disclosure, the Complete Assembled Device is comprised of a single microscale compartment, a first microscale channel which serves as an inlet for liquid culture medium, a second microscale channel which serves an an outlet for liquid culture medium, and primary human hepatocytes that are immobilizd in a matrix material comprised of alginate and configured as a microscale scaffold. The CAD including its constituent biological materials is perfused with a liquid culture medium. Subsequently, a medium suitable for cryopreservation of cells (e.g., 90% fetal bovine serum, 10% DMSO) is introduced into the microscale channel that serves as the fluid inlet and is permitted to flow in and around the biological materials until the liquid culture medium is replaced by the liquid cryopreservation medium. The Complete Assembled Device is frozen thus cryopreserving the primary human hepatocytes. The CAD is then maintained and stored in its cryopreserved state for a period of time. At an appropriate subsequent time, liquid is introduced into the microscale channel that serves as the fluidic inlet and is permitted to flow in and around the biological materials that comprise the Complete Assembled Device (thus serving as a liquid culture medium), and then to exit the microscale chamber through the microscale channel that serves as the fluidic outlet, and to recirculate repeatedly through the CAD until such time as the primary human hepatocytes have been found to have partially, substantially, or totally recovered at least one attribute or function of the biological activity the cells possessed prior to storage, for example the metabolic enzyme activity characteristic of primary human hepatocytes functioning in vivo.

In another embodiment of the disclosure, the Complete Assembled Device is comprised of a first microscale compartment, a first microscale channel which serves as an inlet for liquid culture medium, a second microscale compartment, a channel connecting the first and second microscale compartments, a third microscale channel exiting from the second microscale compartment and serving as an outlet for liquid culture medium, and primary rat hepatocytes that have been previously hypothermically preserved, cold-preserved or cryopreserved and that are cultured in the first microscale chamber in an extracellular matrix material composed of collagen gel. The second microscale chamber contains cellular material drawn from a different mammalian organ. Liquid comprised of fetal bovine serum and containing certain additional proteins such as albumen is introduced into the microscale channel that serves as the fluidic inlet and is permitted to flow in and around the biological materials until such time as the entire cell culture system is filled with liquid. The liquid is then allowed to rest in the cell culture system without any circulation for a period of one hour. At the end of that time the housing of the device is opened and rat primary fibroblasts are added to the first compartment. The housing of the device is then closed again and the liquid is permitted to recirculate in the device for one hundred forty-four hours, at which time the primary human hepatocytes and fibroblasts have been found to have recovered some of the metabolic enzyme activity and expression levels characteristic of those cell types functioning in vivo. This embodiment is not intended to limit the variety of embodiments the disclosure can manifest but rather to illustrate one of the many permutations and combinations of sequential steps, device features, preparations of biological materials, and particular methods of application of the culture medium, that may comprise the disclosure and its use.

Business Methods. In one embodiment, the disclosure provides for a method of running a business wherein customers (such as scientific researchers, commercial or industrial toxicologists, physicians, chemists, biochemists, or other practitioners) may benefit from:

(a) the convenience of being able to perform experiments utilizing biological materials that have already been cultured, preserved, maintained, stored, and/or transported to the customer on, or as an integral part of, the same physical substrate that the customers will subsequently utilize to perform their experiment or procedure, without first having to culture and prepare the biological materials themselves (i.e., the Complete Assembled Device); and

(b) with respect to the experiments, tests or procedures that the customers may utilize one or more features of the present disclosure to perform on biological material, enhanced or advantageous results being achieved due to the fluid culture medium having come into direct contact with the biological material while the culture medium was in a condition of moving or flowing (a “flow condition”) in at least one prior instance or interval during which the biological material was being cultured, preserved, maintained, stored, or transported, or while at least one physiological attribute or function of the biological material was being recovered or reconstituted after said attribute or function had been compromised or had diminished during or prior to any of the foregoing. This prior interaction of culture medium with biological material under the flow condition causes at least one of the biological materials' physiological attributes or functions to be in an improved state during the subsequent experiment or procedure, which in turn causes the biological materials and the CAD to produce advantageous or enhanced experimental results.

For the purpose of description, “flow condition” can mean flow or movement of the culture medium above and beyond any movement or flow that might occur while the apparatus in which biological materials are cultured or maintained is initially perfused or loaded with culture medium, is subsequently replenished with additional culture medium or additives, or is depleted by sampling. In one embodiment, flow condition may or may not include movement due to vibration, centrifugation, stirring, and/or shaking. In some embodiments of practice of the disclosure, a flow condition may again occur, or may continuously endure, during the stage of utilization of the disclosure in an actual experiment or procedure, subsequent to the time the flow condition occurred during the prior culture, preservation, maintenance, storage, transportation, or recovery or reconstitution of the at least one physiological attribute or function of the biological materials.

For the purpose of description, “physiological attribute or function” can mean any attribute or function of any biological material, whether naturally occurring, man-made, or otherwise synthetic or not naturally occurring, which attribute or function influences, determines, regulates, controls, or comprises any factor, state, activity, manifestation, expression, or outcome that a user of at least one CAD may wish to measure, cause, maintain, control, regulate, effect, limit, promote, inhibit, express, or exploit.

Because the biological material has been in contact with the culture medium moving under a condition of flow, the customer may expect that at least one of the physiological characteristics or functions of the biological materials may be improved. “Improved” in the foregoing sentence may mean that the biological materials, which are cultured and maintained in vitro in the disclosure, more closely emulate at least one material physiological characteristic as they evidence it in vivo. For example, having previously been in contact with culture medium in a flow condition during their culture, preservation, reconstitution, etc., the biological materials may manifest levels of viability, genetic or metabolic competency, or inter- or intra-cellular signalling closer to the levels at which those phenomena may be found in vivo. Alternatively, “improved” may mean that exaggerated or particularly desired states of function or productivity may be more effectively induced in the biological materials. Alternatively, “improved” may mean that the biological materials are able to more advantageously manifest any characteristic or function that may be of interest to the customer, than they would have been able to manifest had they not been exposed to the fluid culture medium under at least one condition of flow during their culture, preservation, maintenance, storage, transportation, reconstitution, or recovery of function, or during the performance of the subsequent experiment or procedure upon them.

In other words, the disclosure affords users higher levels not only of operational convenience, but also of improved functionality of the biological materials cultured in vitro within it, compared to what that functionality would have been had the biological materials not been exposed to the culture medium under a flow condition at least once during the prior times of culture, preservation, maintainance, storage, transport, or reconstitution or recovery of at least one desired physiological property, of the biological material. (A flow condition may also resume or continue during the actual performance of the experiment, diagnostic test, therapeutic treatment, bioreactor operation, or other procedure upon the biological materials, which resumption or continuation may provide further beneficial impact on the results of the experiment or operation performed.)

To facilitate the CAD's compatibility with automated materials handling equipment and other standard laboratory equipment, the CAD may be configured in various sizes and scales within the general range of microscale or larger devices, and it may also be multiplexed so that a plurality of CADs may be configured for parallel, simultaneous operation on a single piece of substrate labware which may be of industry-standard dimension, such as a 12-well, 96-well, or 384-well, or other-scale microtiter plate.

In one embodiment, the CAD may comprise one element of a system also comprising at least one other element (an “integrated system”), the other elements of which system may comprise such equipment and materials as: at least one pump; at least one fluid culture medium; at least one reservoir to hold a quantity of culture medium sufficient to operate in the CAD and possibly greater than the CAD can directly, physically hold or contain at any one moment in time; microscale tubing to conduct the fluid culture medium between and among the CAD and the at least one other element of the system; at least one debubbler to remove bubbles from the fluid flow path of the culture medium; at least one media conditioner; at least one microfluidic interface to a bioanalytic device; at least one sensor to generate a signal in response to at least one status of the CAD, the biological materials, or the culture medium; at least one signal transducer to generate from the signal received from the sensor electronic information receivable by a computer, microprocessor, or other electronic device (which transducer may be integral either to the sensor or to said computer, microprocessor or other electronic device); a computer, microprocessor, or other electronic device to receive the signal from the sensor/transducer, to process and/or analyze that signal, to store the information so analyzed, to communicate that information, and/or other information, to remote devices or locations via the internet or other means of public or private utility or communication, and/or to receive information from such remote devices or locations via the internet or other means of public or private utility or communication; to receive, process, analyze and/or store signals or information from at least one other element of the system, and to send signals or information to at least one element of the system for the purpose of controlling the operation or state of that at least one element, or of controlling at least one condition of the biological materials. In a preferred embodiment, an element of the system may also comprise a housing, chamber, or enclosure in which the CAD and possibly other elements of the system are enclosed for purposes of climate control, sequestration from sources of contamination, instrumentation convenience, and the like.

In one embodiment, at least one interface to a bioanalytic device may be a microfluidic interface and it may interface to a mass spectroscopy device, a high-pressure liquid chromatography device, to a camera, to a microplate reader, or to other devices useful for analyzing the products and outcomes of biological experiments, operations and procedures.

In one embodiment, at least one media conditioner may comprise a heater and/or cooler to modify the temperature of the fluid culture medium; a mechanism for gassifying or degassifying the fluid culture medium; a mechanism for changing the volume or biochemical composition of the fluid culture medium, or for adding materials or substances to it; or a sampling port for extracting fluid culture medium from the system.

In one embodiment, the method of operating the business provided by the disclosure may comprise offering some or all of the features of the disclosure for commercial or non-commercial use in conjunction with also offering at least one or more of the at least one other elements of an integrated system. The practitioner of the disclosure may sell, license, or otherwise purvey Complete Assembled Devices, as well as integrated systems and/or other instrumentation for the customer to operate in conjunction with the Complete Assembled Devices through one or more appropriate business arrangements which the practitioner of the disclosure may advantageously enter into with the providers of the at least one other elements.

A practitioner of the disclosure may, alternatively, utilize the at least one CAD, in conjunction with at least one other element of the system described above, to offer at least one commercial or non-commercial service. For example, the practitioner of the disclosure might operate its own laboratory facility, utilizing CAD's in which the practitioner had previously cultured, preserved, and stored biological materials, reconstituting those biological materials at such later time as a customer had provided test substrate materials to the practitioner's laboratory and contracted for experiments or other procedures to be performed upon them.

In one embodiment of the business methods enabled by the disclosure, the practitioner of the disclosure may provide to his or her customer an integrated system comprising at least one Complete Assembled Device (“CAD”) with biological materials pre-cultured and stabilized within it; an instrument configured to accommodate and operate upon the at least one CAD and also comprising at least one other element of the integrated system, such as a pump or a microprocessor with connection to the internet; as well as supplies of other, associated disposable materials such as culture medium, microfluidic tubing, and experimental reagents. The CAD would be delivered (transported) to a location specified by the customer and stored there until the customer was ready to use the CAD. At the appointed time the customer would use the CAD in conjunction with culture medium and the instrument to bring the culture medium into direct contact with the biological materials inside the CAD under a flow condition, and thereby partially, substantially or totally recover or reconstitute at least one physiological attribute or function of the biological material that had previously been compromised or had diminished. Having recovered, restored, or otherwise enhanced or improved at least one aspect or degree of the desired functionality of the biological materials, the customer would then introduce a test substrate of interest into the culture medium and perform an experiment or procedure on the integrated system, exposing the test substrate to the biological materials inside the CAD.

In one embodiment, CAD's may be fabricated in materials that are disposable, such as polyester or plastic, providing the customer the convenience of not having to perform the time-consuming and often difficult operations of thoroughly cleaning biological matter from non-disposable labware so that the labware can be re-used. Because the customer will be able to receive from the practitioner of the disclosure biological materials that are already stored and transported, under quality-controlled processes, directly upon the culture device within which the experiment will occur, the biological materials will require only a step of reconstitution to be performed by the customer to make the device fully ready for use. (However, the customer may frequently introduce into the CAD additional biological materials specific to the experimental design or to the interests or preferences of the researcher. Such customization would add more steps and effort to the process of preparing an experiment.)

Manufacture of the Complete Assembled Device (“CAD”) including preservation of biological materials within it. The Complete Assembled Device typically comprises an aggregation of separate elements, e.g., at least one chambers, channels, inlets, or outlets, which when appropriately mated, joined, or otherwise integrated together, form the culture device of the disclosure. Preferably these elements are provided in an integrated, “chip-based” format. The CAD also comprises biological materials which may be cultured directly in the culture device, or which in certain embodiments may be cultured in a three-dimensional (“3D”) configuration in an extracellular matrix material such as a calcium alginate scaffold, which extracellular matrix material may in turn be placed, affixed, adhered, inserted, directly formed, or otherwise positioned or maintained within a compartment or other feature of the culture device. The CAD also comprises a housing that encloses the culture device and the biological materials, which housing may in turn comprise at least one fluidic inlet and at least one fluidic outlet.

In one embodiment, the bottom portion can comprise a solid substrate that is substantially planar in structure, and which has at least one substantially flat upper surface. A variety of substrate materials may be employed as the bottom portion, including silicon (glass). Because the devices can be microfabricated, substrate materials might be selected based upon their compatibility with known microfabrication techniques, e.g., photolithography, thin-film deposition, wet chemical etching, reactive ion etching, inductively couple plasma deep silicon etching, laser ablation, air abrasion techniques, injection molding, embossing, and other techniques.

In other embodiments, the substrate materials can comprise polymeric materials, e.g., plastics, such as polystyrene, polymethylmethacrylate (PMMA), polycarbonate, polytetrafluouroethylene (TEFLON®), polyvinylchloride (PVC), polydimethylsiloxane (PDMS), polysulfone, and the like. Such substrates are readily manufactured from masters, using well-known molding techniques, such as injection molding, embossing or stamping, or by polymerizing the polymeric precursor materials within the mold. Such polymeric substrate materials are preferred for their ease of manufacture, low cost and disposability, as well as their general inertness to extreme reaction conditions. Again, these polymeric materials may include treated surfaces, e.g., derivatized or coated surfaces, to enhance their utility in the system, e.g., provide enhanced fluid direction, cellular attachment or cellular aggregation.

In one embodiment of the present disclosure, the channels and/or chambers of a culture device are typically fabricated, using the above-described techniques, as grooves or indentations configured into the upper surface of the substrate, i.e., in the top surface of the bottom portion of the culture device. The lower surface of the top portion of a culture device, which top portion typically comprises a second planar substrate, is then overlaid upon and bonded to the surface of the bottom substrate, sealing the channel and/or chambers (i.e., the interior portion) of the device at the interface of these two components. Bonding of the top portion to the bottom portion may be carried out using a variety of known methods, depending upon the nature of the substrate material. For example, in the case of glass substrates, thermal bonding techniques may be used which employ elevated temperatures and pressure to bond the top portion of the device to the bottom portion. Polymeric substrates may be bonded using similar techniques, except that the temperatures used are generally lower to prevent excessive melting of the substrate material. Alternative methods may also be used to bond polymeric parts of the device together, including acoustic welding techniques, or the use of adhesive, e.g., UV curable adhesives, and the like.

In one embodiment of the present disclosure, the biological materials comprise a porous calcium alginate scaffold in which cellular or subcellular materials, such as hepatocytes, are immobilized and cultured. The scaffold, sometimes called a sponge, may be formed by preparing a solution of lyophilized (“freeze-dried”) alginate of specific concentration, and subsequently combining therewith a calcium gluconate solution, also of specific concentration. The physical size and shape of the resulting calcium alginate hydrogel (its external dimensions) may be configured by cutting with a blade or with a pre-formed die, or by other means.

The fluidics used to actuate the flow of fluid into, through, or out of a CAD are appropriately scaled for the size of the device. In a chip-based format, the fluidic connections may be “microfluidic,” e.g., a fluidic element, such as a passage, chamber or conduit has at least one internal cross-sectional dimension, e.g., depth or width, of greater than or equal to one tenth of one μm (0.1 μm), and less than one cm (1 cm). In one embodiment of the present disclosure, the channels connecting the chamber of the culture device typically include at least one microfluidic channel. In another embodiment of the present disclosure, none of the features of the culture device contain microfluidic channels.

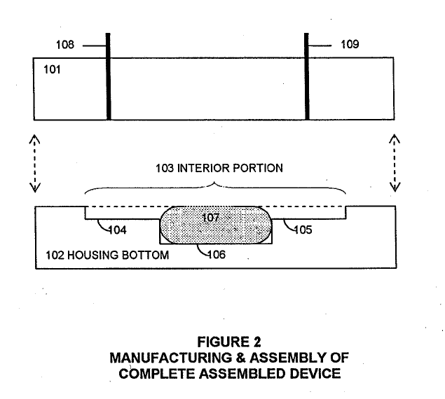

FIG. 2 schematically represents, in cross-section view, a prophetic embodiment of the disclosure illustrating the manufacture and assembly of the Complete Assembled Device, and its subsequent operation to culture biological materials and preserve them for storage until their eventual reconstitution and use. In this embodiment, the housing top 101 and the housing bottom 102 are fabricated in acrylic plastic; the bottom surface of the top 101 and the top surface of the bottom 102 are configured to be substantially planar to assure a tight seal that does not permit fluid leakage when the top 101 and bottom 102 are mated together. The interior portion of the microscale culture device 103, comprising at least one chamber 106, at least one channel with integrated fluidic inlet 104 connecting fluidically to the at least one chamber 106, at least one channel with integrated fluidic outlet 105 connecting fluidically to the chamber 106, is configured integrally in the housing bottom 102 through a process of embossing (stamping) the acrylic plastic substrate. Penetrating the housing top 101 are fluidic interconnects 108, 109 that provide conduits to microbore tubing that connects the CAD to other elements of the integrated system including without limitation the at least one pump (not shown) and the at least one fluidic reservoir (not shown). When the housing top 101 and the housing bottom 102 are mated together, the fluidic interconnect 108 delivers fluid culture medium into the fluidic inlet with integrated channel 104; and the fluidic interconnect 109 receives fluid culture medium, driven by the pump (not shown) under positive hydraulic pressure, from the channel with integrated fluidic outlet 105. The fluidic interconnects 108, 109 are comprised of stainless steel, microbore cannulae that have been press-fitted through holes drilled for the purpose through the housing top 101. Inside the chamber 106 is biological material comprised of porous alginate scaffold 107 in which have been immobilized primary human hepatocytes (not shown). The hepatocytes have been permitted to permeate the porous scaffold 107, become immobilized within its topography, and cluster together over a period of forty-eight hours while perfused with culture medium, forming spheroids of cells within the scaffold 107.

At this time the housing top 101 and the housing bottom 102 are mated together to form a tight seal, using external clamps (not shown). (In alternative embodiments, other means of mechanically joining the housing top 101 and the housing bottom 102 may be employed. For example, they may be bolted together by screws.) The biological material comprising both extracellular matrix material (the scaffold 107) and cellular material (the primary human hepatocytes clustered in spheroids (not shown) immobilized inside the scaffold 107) is thus completely enclosed inside the sealed housing. Mating the housing top 101 and housing bottom 102 together creates the CAD and a completely self-contained microfluidic pathway within it, which microfluidc pathway comprises the fluidic interconnect 108, the integrated inlet and channel 104, the chamber 106, the integrated channel and outlet 105 and the fluidic interconnect 109. The CAD is then integrated with the instrument through microfluidic tubing which is attached to the external ends of the fluidic interconnects 108, 109, and which gives effect to the recirculatory flow of culture medium into, through, and out from the CAD. The CAD and the instrument are then introduced into a controlled-rate freezer (not shown), and the instrument's pump is actuated, causing culture medium to flow through and around the biological materials while they are undergoing cold-preservation. Once the process of cold preservation is complete, the instrument is removed from the controlled-rate freezer, the CAD is detached from the instrument, and the CAD is then placed inside packaging that provides mechanical protection during storage (not shown). Thereafter, the CAD is maintained at a temperature of 4° C. until such time as the functionality of the biological materials needs to be recovered and reconstituted in preparation for an experiment or other procedure to be performed on the CAD and the instrument.

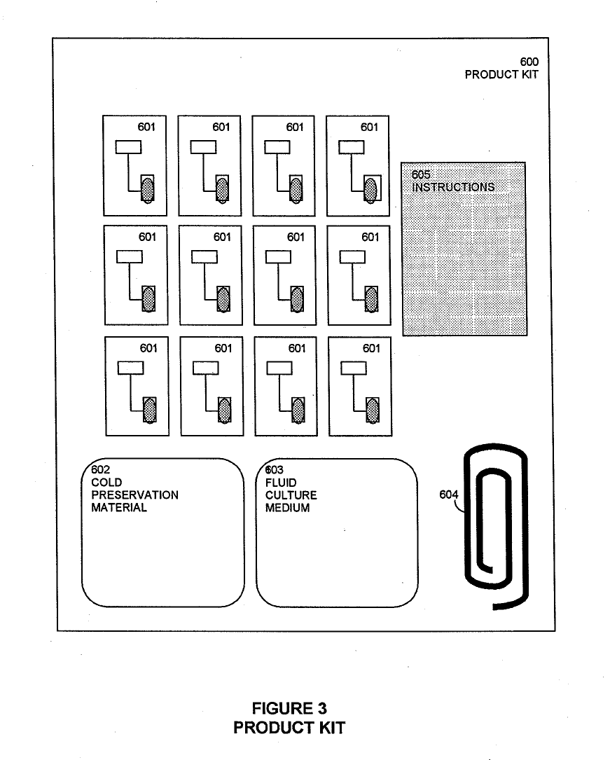

Embodiment in product kits. As illustrated in FIG. 3, the disclosure may be embodied in a product kit 600 that is provided to the customer. In one embodiment, the kit 600 is envisioned to contain the at least one Complete Assembled Device 601 including the biological materials already preserved, stored and maintained within it, which the user or customer would receive in a single package. The kit might also include appropriate quantities of cold preservation material 602, such as dry ice, to maintain the successful preservation of the biological materials during their transportation and storage and prior to their use by the researcher. The kit would also separately include an appropriate quantity of liquid culture medium 603 for the customer to use in reconstituting the one or more desired functions or attributes of the biological materials, and for operating the subsequent experiment(s). The kit would also include a supply of disposable microbore tubing 604 for the user to attach to the inlets and outlets in the housing of CAD 601. The kit would also include instructions 605 on how to operate the Complete Assembled Device 601 in conjunction with at least one instrument (provided separately and not shown in FIG. 3) so as to reconstitute the at least one attributes and functions of the biological materials and, subsequently, so as to successfully perform an experiment.

In a different embodiment of the kit, the biological materials of the kit would comprise microscale calcium alginate scaffolds in which cellular products had previously been immobilized, cultured and preserved by the practitioner of the disclosure, and transported to the user in that form. The kit would also separately include at least one microscale culture device and housing, as well as a quantitiy of culture medium, materials such as dry ice to maintain cold-preserved or cryopreserved conditions, disposable ancillary materials, and instructions. At the researcher's (the user's) own premises, the researcher would mechanically place the scaffold, laden with its attached, immobilized cells, into the at least one chamber of the culture device and then seal up the housing, creating the at least one Complete Assembled Device. The researcher would then add culture medium, attach the Complete Assembled Device to an at least one instrument, and then proceed to operate the CAD in conjunction with the instrument first to recover or reconstitute at least one desired attribute or function of the biological material that comprises the CAD, and then to run an experiment or other procedure utilizing the biological material that comprises the CAD.

In some embodiments of the present disclosure, attachment of the biological material to the CAD can be achieved in any one of many different ways, some of which are described herein. Such prepared biological material can be preserved, transported, and stored in the CAD until it is ready for use. For example, the biological material can be in a frozen state, or in a cold-preserved or hypothermic state, in the CAD until ready for use.

As described herein, reconstitution of the biological material for use can be achieved by allowing interaction of the biological material with a fluidic system. In one embodiment, one or more fluids can be circulated with respect to the material to allow warming of the biological material from the frozen or hypothermic state to a desired state for performing assays.

Although the above-disclosed embodiments have shown, described, and pointed out the fundamental novel features of the invention as applied to the above-disclosed embodiments, it should be understood that various omissions, substitutions, and changes in the form of the detail of the devices, systems, and/or methods shown may be made by those skilled in the art without departing from the scope of the invention. Consequently, the scope of the invention should not be limited to the foregoing description, but should be defined by the appended claims.

What is claimed is:

1. A culture device comprising at least one microscale feature that is configured to advantageously effect the culture, preservation, maintenance, storage, or transportation of biological materials, or the partial or total recovery or reconstitution of at least one material attribute or function that the biological material previously possessed, by providing for the biological materials to be brought into direct contact with a fluid culture medium that operates under a condition of flow in the culture device.
